(19) United States
(12) Patent Application Publication
(10) Pub. No.: US 2026/0301852 A1
(43) Pub. Date: Oct. 1, 2026

(54) RECOGNITION PHYSICS BASED SYSTEM AND METHOD FOR SIMULATING AND PREDICTING BIOLOGICAL PROCESS DYNAMICS

(71) Applicant: Jonathan Washburn, Austin, TX (US)

(72) Inventor: Jonathan Washburn, Austin, TX (US)

(21) Appl. No.: 19/305,532

(22) Filed: Aug. 20, 2025

Related U.S. Application Data
(60) Provisional application No. 63/778,998, filed on Mar. 27, 2025.

(51) Int. Cl. G16B 5/00

(52) U.S. Cl. CPC G16B 5/00

(57) ABSTRACT

A system and method for simulating the time-dependent dynamics of biological processes involving DNA are disclosed. The invention is based on Recognition Physics (RS) principles and the DNA Recognition Physics (DNARP) framework, in which a DNA state is represented as a triplet of sequence, shape, and energy components. The system initializes an RS-defined state, applies RS-based evolution rules, and computationally evolves the state using time-dependent governing constructs including a DNA Lagrangian, a DNA operator, and a DNA Recognition Transform. Functional outputs such as gene expression rate, coherence levels, or regulatory activity are predicted without reliance on empirical parameter fitting. The computational system comprises an input module, a dynamic simulation engine configured to apply RS constructs, and an output module that generates time-series data. The invention enables parameter-free prediction of biological dynamics, including gene regulation, drug response, circadian rhythms, and DNA repair processes, based on first principles.

### CROSS-REFERENCE TO RELATED APPLICATIONS

This application claims the benefit of priority to U.S. Provisional Patent Application No. 63/778,998, filed Mar. 27, 2025, the entirety of which is incorporated herein by reference.

### STATEMENT REGARDING FEDERALLY SPONSORED RESEARCH OR DEVELOPMENT

Not applicable. The invention described herein was not made with government support, and no governmental rights apply.

### NAMES OF THE PARTIES TO A JOINT RESEARCH AGREEMENT

Not applicable.

### REFERENCE TO A SEQUENCE LISTING, A COMPUTER PROGRAM LISTING, OR A LARGE TABLE APPENDIX

Not applicable. No sequence listing, computer program listing appendix, or large table appendix is submitted with this application.

### FIELD OF THE INVENTION

The present invention relates generally to computational modeling of biological systems. More particularly, it concerns a physics-based system and method for simulating and predicting time-dependent biological process dynamics, including DNA regulatory interactions, using parameter-free constructs derived from Recognition Physics.

### BACKGROUND OF THE INVENTION

Understanding the dynamics of biological processes, including gene regulation, metabolic adaptation, cellular response to environmental stimuli, and circadian rhythms, is a central challenge in medicine and biotechnology. Predictive modeling of such processes is essential for applications ranging from drug development and personalized medicine to synthetic biology and systems-level analysis of living organisms.

Conventional computational approaches generally rely on kinetic models formulated as systems of ordinary or partial differential equations. These models depend on empirically measured parameters such as reaction rates, binding affinities, and molecular concentrations. While such models can reproduce observed dynamics within the range of calibration data, they often require extensive parameter fitting and may lack predictive power outside the training conditions.

Existing frameworks frequently treat DNA as a static blueprint, or incorporate its dynamics through simplified mechanical or statistical approximations. These methods rarely account for deeper physical principles such as coherence phenomena, quantum-level energy interactions, or the integrated role of sequence, structural conformation, and energetic state. As a result, their capacity to model time-dependent changes in DNA function and regulation remains limited.

Moreover, heavy reliance on adjustable parameters reduces reproducibility and generalizability across different biological contexts. Predictive capability is constrained by the availability of high-quality experimental datasets for calibration, and extrapolation beyond observed conditions is typically unreliable. This limits the usefulness of existing models in scenarios requiring first-principles prediction, such as novel drug interactions, genetic perturbations, or environmental stresses outside laboratory calibration ranges.

Accordingly, there is a need for a unified, physics-based framework that models the time-dependent dynamics of biological processes, particularly those involving DNA, from first principles. Such a framework should reduce or eliminate reliance on empirical parameterization, incorporate sequence, shape, and energy dynamics in an integrated manner, and provide predictive power grounded in universal physical laws.

Recognition Physics (RS), introduced by the inventor, addresses this need by deriving governing constructs from a foundational axiom without free parameters. Building upon this foundation, the DNA Recognition Physics (DNARP) framework encodes DNA states as a triplet of sequence, shape, and energy components. Extending DNARP to dynamic biological processes provides a basis for parameter-free simulation of DNA dynamics and related biological functions, thereby overcoming the limitations of conventional approaches.

### SUMMARY OF THE INVENTION

The present invention provides a system and method for simulating and predicting the time-dependent dynamics of biological processes based on Recognition Physics (RS). Unlike conventional kinetic models that rely on empirical parameters, the invention derives governing constructs from RS axioms, enabling parameter-free or minimally parameterized simulations grounded in universal physical principles.

In one aspect, the invention provides a method for simulating a biological process involving a DNA molecule. The method comprises: (a) defining an initial DNA Recognition Physics (DNARP) state D(t0)=(S(t0), H(t0), E(t0)), where S represents sequence, H represents shape, and E represents energy; (b) defining one or more RS-based evolution rules specifying how at least one of S, H, or E changes over time in response to simulated biological interactions or stimuli; (c) evolving the state D(t) over a time interval by iteratively solving time-dependent RS constructs, including a DNA Lagrangian LDNA(t), a DNA operator H\hat{ }DNA(t), and a DNA Recognition Transform FDNA(E(t)), using RS-derived constants; and (d) predicting the time course of one or more functional outputs, such as gene expression rate, protein concentration, or coherence level.

In another aspect, the invention provides a computational system for carrying out the above method. The system comprises: (a) an input module for defining the initial DNARP state and RS-based evolution rules; (b) a dynamic simulation engine configured to evolve the state D(t) using RS constructs and constants without reliance on free parameter fitting; and (c) an output module for storing and presenting predicted time-dependent properties and functional outcomes.

The invention enables parameter-free modeling of biological dynamics with improved predictive capability compared to conventional approaches. By grounding the simulation framework in RS, the system integrates sequence accessibility, conformational changes, and coherence state dynamics in a unified manner. This approach provides a robust foundation for modeling gene regulation, response to drug exposure, circadian rhythms, DNA repair, and other time-dependent biological processes.

Advantages of the invention include elimination of extensive parameter fitting, reproducibility across biological contexts, predictive power outside calibration ranges, and direct mapping to experimentally measurable outputs. The system and method thereby provide a first-principles platform for computational biology that overcomes the limitations of existing kinetic and statistical models.

### BRIEF DESCRIPTION OF THE DRAWINGS

FIG. 1 illustrates the DNA Lagrangian LDNA stability functional (100), showing the stability variable C(r) (102), the periodicity parameter P (104), and constants κDNA (106) and λDNA (108).

FIG. 2 illustrates the DNA operator H\hat{ }DNA coherence construct (110), showing the position variable x (112), operator term d/dx (114), constant XDNA (116), and coherence factor kDNA (118).

FIG. 3 illustrates the DNA Recognition Transform FDNA(E) (120), showing the stabilization factor Sstab (122), energy level En (124), coherence constant Ecoh (126), and periodicity parameter P0 (128).

FIG. 4 illustrates the time-dependent Schrödinger-like equation (130), showing the quantum state ψ(x,t) (132), reduced Planck constant h-bar (134), and the operator H\hat{ }DNA(t) (136).

FIG. 5 illustrates the approximate propagation step (140), showing the exponential operator e\hat{ }(−iH\hat{ }DNAΔt/h-bar) (142) and evolved state ψ(t+Δt) (144).

FIG. 6 illustrates the time-dependent DNA Recognition Transform FDNA(E(t)) (150), showing the stabilization factor Sstab(t) (152), energy level En(t) (154), coherence constant Ecoh (156), and periodicity parameter P0 (158).

FIG. 7 illustrates the relation between coherence state index n(t) (160), base rate R0 (162), and resulting output rate R(t) (164).

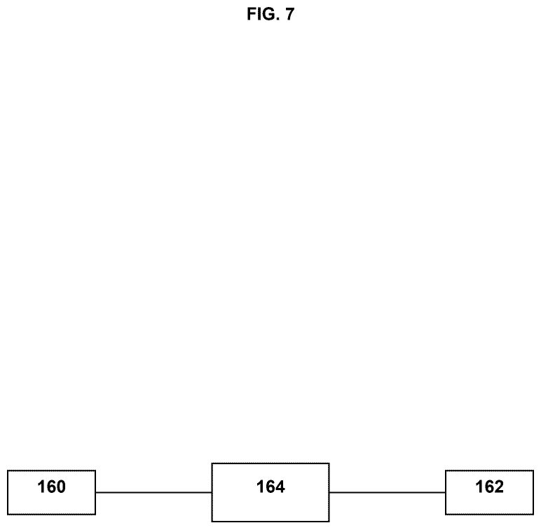

FIG. 8 illustrates a system architecture diagram (170) showing the input module (172), dynamic simulation engine (174), and output module (176).

FIG. 9 illustrates a method flowchart (180), showing initialization of the DNARP state (182), definition of RS-based rules (184), time evolution loop (186), and generation of predicted outputs (188).

FIG. 10 illustrates a schematic of DNA dynamics (190), showing sequence accessibility S(t) (192), DNA shape H(t) (194), and coherence energy E(t) (196) as they evolve over time.

FIG. 11 illustrates an example application of gene activation (200), showing an activator molecule (202) binding to DNA (204), a resulting transition from coherence state E1 (206) to E2 (208), and the corresponding increase in expression rate (210).

### LIST OF REFERENCE NUMERALS

100—DNA Lagrangian LDNA stability functional
102—Stability variable C(r)
104—Periodicity parameter P
106—Constant κDNA
108—Constant λDNA
110—DNA operator H\hat{ }DNA coherence construct
112—Position variable x
114—Operator term d/dx
116—DNA length constant XDNA
118—Coherence factor kDNA
120—DNA Recognition Transform FDNA(E)
122—Stabilization factor Sstab
124—Energy level En
126—Coherence constant Ecoh
128—Periodicity parameter P0
130—Time-dependent Schrödinger-like equation
132—Quantum state ψ(x,t)
134—Reduced Planck constant h
136—Time-dependent DNA operator H\hat{ }DNA(t)
140—Approximate propagation step
142—Exponential operator e\hat{ }(−iH\hat{ }DNAΔt/h)
144—Evolved state ψ(t+Δt)
150—Time-dependent DNA Recognition Transform FDNA(E(t))
152—Time-dependent stabilization factor Sstab(t)
154—Time-dependent energy level En(t)
156—Coherence constant Ecoh
158—Periodicity parameter P0
160—Coherence state index n(t)
162—Base rate R0
164—Output rate R(t)
170—System architecture
172—Input module
174—Dynamic simulation engine
176—Output module
180—Method flowchart
182—Initialization step
184—Definition of evolution rules
186—Time evolution loop
188—Output prediction
190—DNA dynamics schematic
192—Sequence accessibility S(t)
194—DNA shape H(t)
196—Coherence energy E(t)
200—Example application of gene activation
202—Activator molecule
204—DNA region
206—Coherence state E1
208—Coherence state E2
210—Expression rate

### DETAILED DESCRIPTION OF THE INVENTION

The invention is grounded in the principles of Recognition Physics (RS), a parameter-free framework derived from the axiom that observation and recognition interactions govern physical systems. Building upon this foundation, the DNA Recognition Physics (DNARP) model encodes the state of a DNA molecule as a triplet D=(S, H, E), where S denotes the sequence component, H denotes the shape component, and E denotes the energy or coherence component. The DNARP triplet is configured to provide a structured representation of DNA states suitable for both static and dynamic modeling.

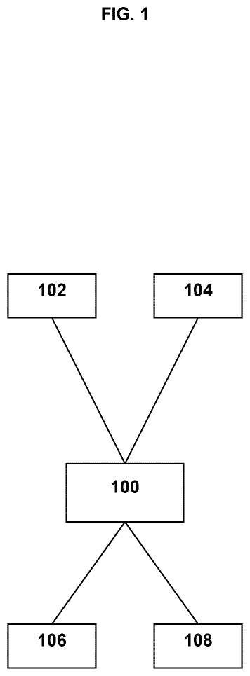

As illustrated in FIG. 1, the DNA Lagrangian LDNA stability functional (100) is configured to govern the structural stability of DNA. The stability variable C(r) (102) evolves according to the periodicity parameter P (104) and is modulated by constants κDNA (106) and λDNA (108). This functional is operative to characterize stable conformations mathematically within RS without reliance on empirical curve-fitting, thereby providing a parameter-free description of DNA stability.

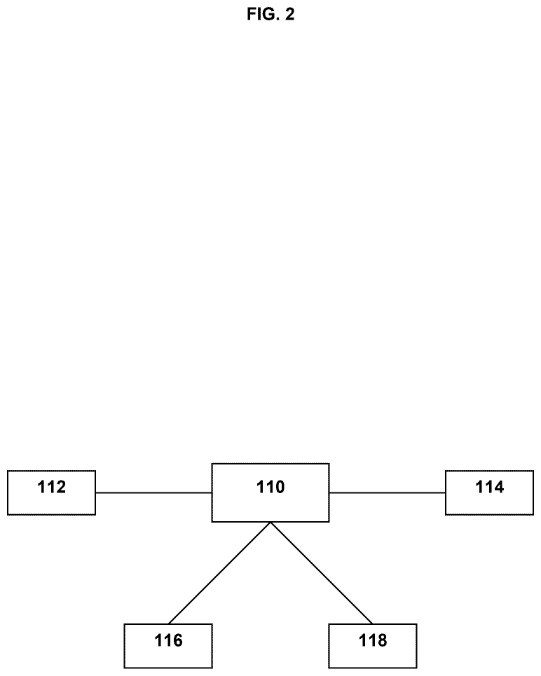

As illustrated in FIG. 2, the DNA operator H\hat{ }DNA coherence construct (110) is configured to govern the quantum-like coherence properties of DNA. The operator includes the position variable x (112) and operator term d/dx (114), and is scaled by the DNA length constant XDNA (116) and the coherence factor kDNA (118). The operator is operative to yield eigenvalues corresponding to discrete coherence levels that define quantized energy states of the DNA molecule.

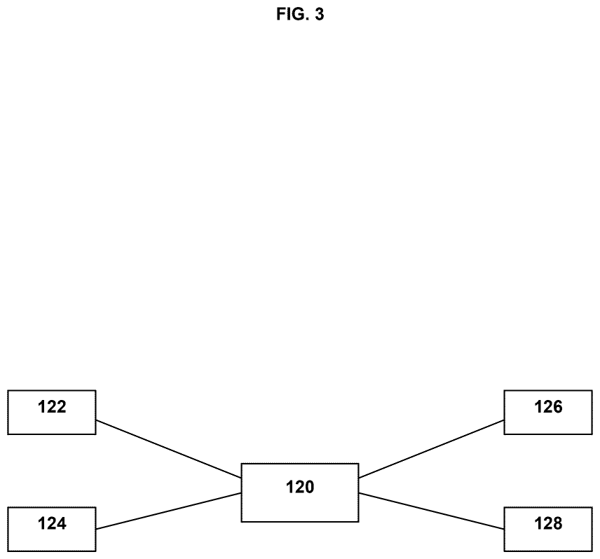

As illustrated in FIG. 3, the DNA Recognition Transform FDNA(E) (120) is configured to map discrete energy levels into measurable amplitudes. The transform is determined by the stabilization factor Sstab (122), energy level En (124), coherence constant Ecoh (126), and periodicity parameter P0 (128). The transform is operative to provide predicted outputs, such as gene expression intensity or coherence state amplitude, from RS-derived energy states.

Together, the constructs LDNA (100), H\hat{ }DNA (110), and FDNA(E) (120) are configured to provide a parameter-free, static description of DNA structure, coherence, and function under the RS framework. These constructs form the foundation for extending DNARP to dynamic biological processes, as described in the following sections.

Biological processes are inherently dynamic, requiring the DNARP framework to be extended from static representations to time-dependent constructs. In the dynamic model, the DNA state is represented as D(t)=(S(t), H(t), E(t)), where each component of the triplet may evolve over time according to RS-based rules that describe interactions with proteins, signaling molecules, environmental factors, or internal feedback.

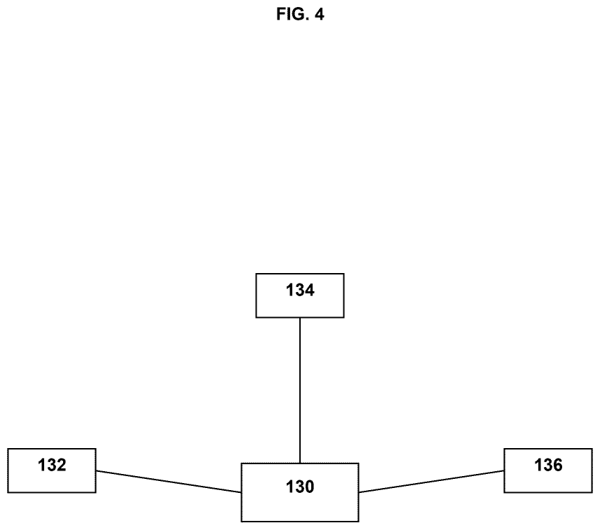

As illustrated in FIG. 4, the time-dependent Schrödinger-like equation (130) governs the evolution of the quantum state ψ(x,t) (132). The operator H\hat{ }DNA(t) (136) acts on the state, with evolution scaled by the reduced Planck constant h-bar (134). This equation defines how DNA coherence states propagate over time under the influence of interactions and external stimuli, establishing a parameter-free dynamic law of motion.

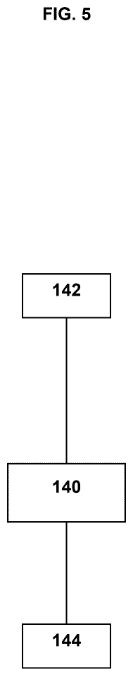

As illustrated in FIG. 5, the approximate propagation step (140) provides a computationally practical means of evolving the state over discrete intervals. The exponential operator e\hat{ }(−iH\hat{ }DNAΔt/h-bar) (142) is applied to advance the state, yielding an updated quantum state ψ(t+Δt) (144). This formulation enables iterative simulation of DNA dynamics across biologically relevant timescales.

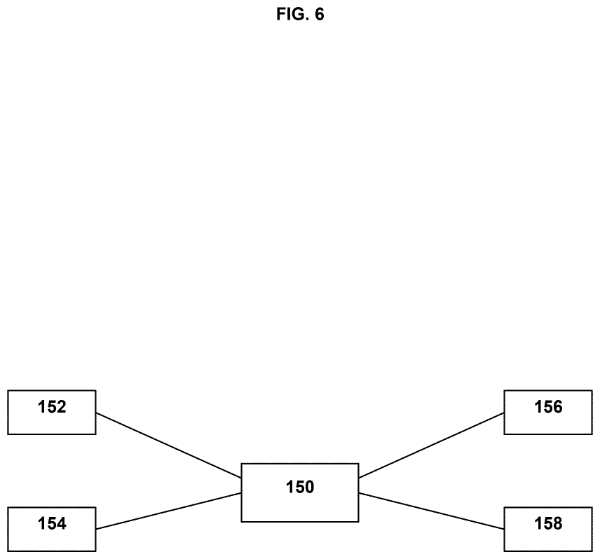

As illustrated in FIG. 6, the time-dependent DNA Recognition Transform FDNA(E(t)) (150) maps evolving energy levels to functional outputs. The transform depends on the stabilization factor Sstab(t) (152), time-varying energy level En(t) (154), coherence constant Ecoh (156), and periodicity parameter P0 (158). This mapping yields time-dependent amplitude outputs |FDNA(E(t))|\hat{ }2 that directly correspond to observable biological effects such as transcription rates, coherence signatures, or regulatory activity.

The constructs LDNA(t), H\hat{ }DNA(t), and FDNA(E(t)) collectively extend DNARP into a dynamic framework. Sequence accessibility S(t) may change due to binding or modification events, shape H(t) may change due to conformational adjustments, and energy E(t) may change through excitation or relaxation processes. These dynamic constructs enable the invention to capture time-dependent biological phenomena using Recognition Physics principles without reliance on empirical parameter fitting.

The invention further provides a method for simulating the dynamics of biological processes using the RS-based DNARP framework. The method is executed computationally and applies the time-dependent constructs described above to iteratively predict biological behavior.

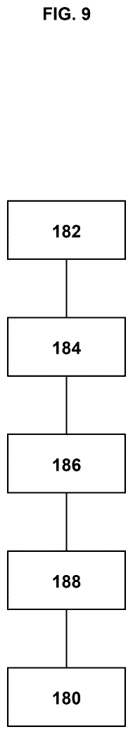

As illustrated in FIG. 9, the method flowchart (180) begins with initialization of the DNARP state (182). In this step, an initial triplet D(t0)=(S(t0), H(t0), E(t0)) is defined based on recognition physics parameters and validated for stability using the static constructs LDNA, H\hat{ }DNA, and FDNA.

The next step involves definition of RS-based evolution rules (184). These rules specify how one or more components of the triplet D(t) will change in response to biological interactions or stimuli. Examples include modification of sequence accessibility S(t) due to repressor binding, conformational change of shape H(t) due to supercoiling, or transition of energy state E(t) due to excitation or relaxation events.

The dynamic simulation then proceeds through a time evolution loop (186). At each time step, the evolution rules are applied to update S(t), H(t), and E(t). The updated values are then substituted into the governing constructs LDNA(t), H\hat{ }DNA(t), and FDNA(E(t)). Where required, the Schrödinger-like equation of FIG. 4 is solved or the propagation step of FIG. 5 is applied to evolve the state ψ(x,t). The result is a continuous or discrete progression of the DNA state over time, reflecting the defined biological context.

The final step of the method is output prediction (188). At each iteration of the time evolution, the functional output is calculated using FDNA(E(t)), producing time-series data such as gene expression rate, coherence intensity, or protein concentration. These predicted outputs provide a parameter-free forecast of biological dynamics.

By integrating initialization, rule definition, time evolution, and output prediction, the method enables computational simulation of DNA dynamics and related biological processes. Importantly, the method does not rely on empirical fitting of kinetic parameters but instead derives from RS-based governing constructs, ensuring reproducibility and predictive accuracy across contexts.

In another aspect, the invention provides a computational system configured to implement the method described above. The system is designed to manage input definitions, perform time-dependent simulation, and generate outputs reflecting predicted biological dynamics.

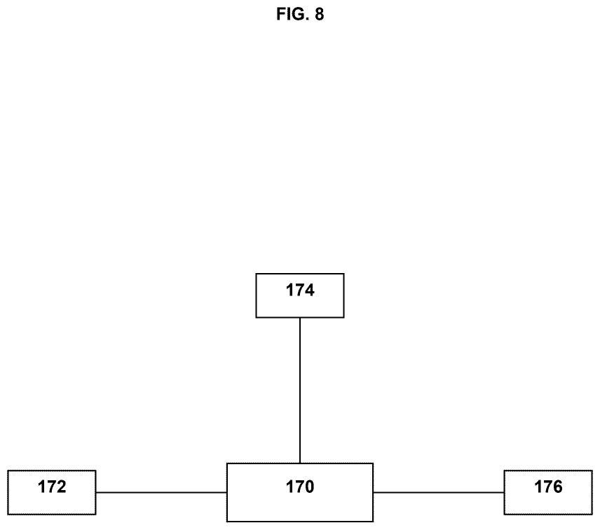

As illustrated in FIG. 8, the system architecture diagram (170) comprises an input module (172), a dynamic simulation engine (174), and an output module (176). These modules are implemented on a processor and memory architecture capable of storing, evolving, and reporting the DNARP state.

The input module (172) enables a user or external software to define the initial DNARP state D(t0)=(S(t0), H(t0), E(t0)) and specify RS-based evolution rules governing its progression. The input module may accept definitions in textual, graphical, or symbolic form, and validates the initial state against static RS constructs to ensure internal consistency.

The dynamic simulation engine (174) forms the core of the system. It applies the defined evolution rules over successive time steps, updating the components of the DNARP state. Within the engine, submodules are responsible for solving the time-dependent DNA Lagrangian LDNA(t), applying the DNA operator H\hat{ }DNA(t), and calculating the DNA Recognition Transform FDNA(E(t)). The engine may implement either exact solutions or approximate propagation steps, such as the exponential operator described in FIG. 5, depending on computational requirements.

The output module (176) stores and presents the predicted results of the simulation. This may include time-series data of sequence accessibility S(t), conformation H(t), energy coherence levels E(t), and associated functional outputs such as transcription rate, coherence amplitude, or regulatory activity. The output module may provide results in graphical form, exportable data files, or integration with external biological modeling platforms.

The system architecture provides a complete framework for parameter-free biological simulation. By coupling input definition, iterative RS-based evolution, and output reporting, the system enables practical deployment of the invention for research, clinical, or industrial applications.

The invention further provides a schematic representation of DNA dynamics, illustrating how the sequence, shape, and energy components evolve under time-dependent conditions. This schematic captures the integrated behavior of the DNARP triplet as it responds to biological interactions or environmental inputs.

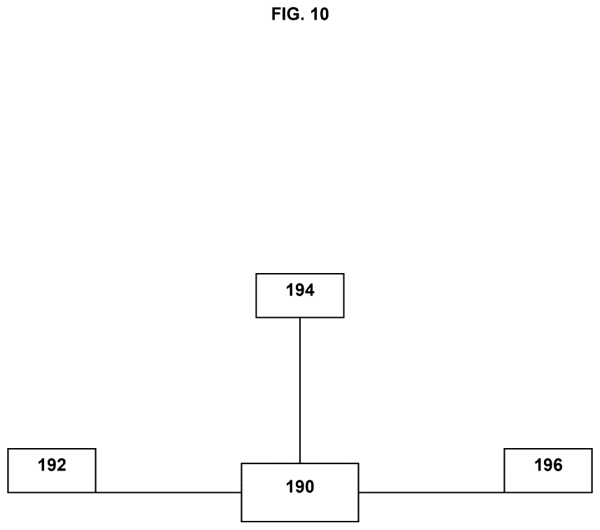

As illustrated in FIG. 10, the DNA dynamics schematic (190) depicts the evolution of sequence accessibility S(t) (192), DNA shape H(t) (194), and coherence energy E(t) (196). These three components are represented as interdependent variables whose values change during the course of simulation.

The sequence component S(t) (192) reflects the accessibility of nucleotide regions. Binding of repressors or activators, or modifications such as methylation, can alter effective accessibility without altering the underlying base sequence. The schematic illustrates how such recognition events modify S(t) during simulation.

The shape component H(t) (194) reflects the conformational state of the DNA molecule. Changes such as supercoiling, groove width variation, or protein-induced bending shift the periodicity and geometric ratios of DNA structure. These conformational changes are incorporated into the simulation as modifications of H(t), as illustrated in the schematic.

The energy component E(t) (196) reflects the coherence state of the DNA system. Excitation by photons, binding events, or stress signals can increase the coherence level, while relaxation processes decrease it. The schematic shows transitions between energy states, which correspond to quantized coherence levels defined by the RS operator constructs.

Together, sequence accessibility S(t) (192), DNA shape H(t) (194), and coherence energy E(t) (196) provide a unified representation of DNA dynamics within the RS-based simulation framework. FIG. 10 illustrates how changes in any one component can propagate to the others, thereby capturing the coupled nature of biological processes.

To illustrate the application of the invention, an example simulation of gene activation is provided. This example demonstrates how the RS-based DNARP framework models the effect of an activator molecule on DNA dynamics and predicts resulting changes in expression rate.

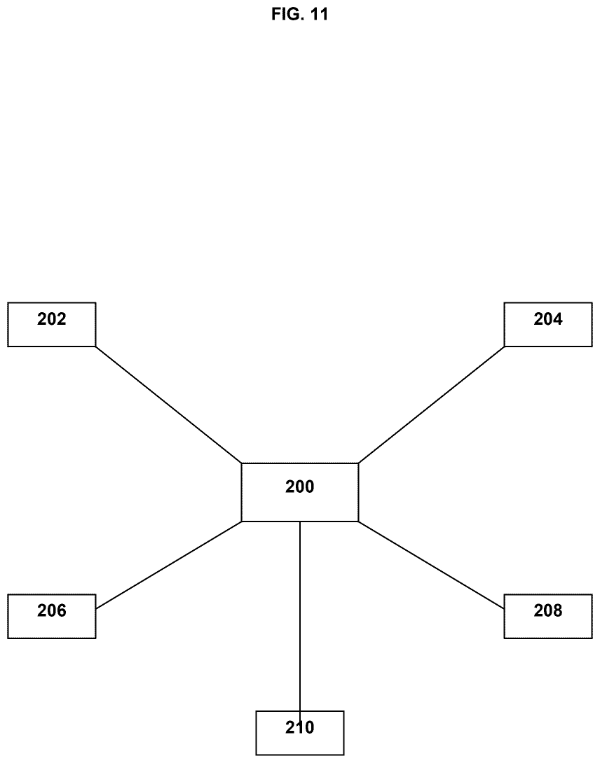

As illustrated in FIG. 11, the example application (200) begins with an activator molecule (202) binding to a DNA region (204). The binding event is defined as an RS-based evolution rule that modifies the potential term within the DNA operator H\hat{ }DNA(t).

Prior to activation, the DNA coherence state is represented as E1 (206), corresponding to a baseline energy of approximately 0.09 electron volts and an expression rate of 50 bases per second. When the activator molecule (202) binds, the rule specifies an induced transition to the higher coherence state E2 (208). This transition doubles the coherence energy to approximately 0.18 electron volts, increasing the rate of base incorporation to 100 bases per second.

The Recognition Transform FDNA(E(t)) maps the updated energy level to an amplitude output, producing an intensity proportional to |FDNA(E2)|². In this example, the calculated value corresponds to a rate increase to approximately 225 bases per second, exceeding the baseline transcription rate by more than fourfold.

A feedback rule may also be applied, such that when the predicted output rate exceeds a threshold of 200 bases per second, the stabilization factor Sstab is increased by 0.5 units. This feedback modifies the system state, influencing subsequent rounds of simulation and demonstrating how the invention captures regulatory loops within biological processes.

The gene activation schematic of FIG. 11 thus illustrates the ability of the invention to model recognition-driven transitions in DNA coherence states and predict their impact on functional outputs. The example confirms that the system provides parameter-free prediction of time-dependent biological dynamics in response to molecular interactions.

In one embodiment, the invention employs a set of RS-derived constants that are defined without reliance on empirical parameter fitting. These constants provide the foundation for all DNARP constructs and ensure parameter-free reproducibility of the invention.

The coherence constant Ecoh (126, 156) is approximately 0.090 electron volts, derived from the golden ratio phi raised to the negative fifth power. Ecoh defines the discrete energy spacing between coherence levels of the DNA system.

The fundamental tick constant tauₒ is approximately 7.33×10⁻¹⁵ seconds when mapped to SI units. Tauₒ represents the minimal recognition interval that bounds temporal evolution within the RS framework.

The golden ratio phi is approximately 1.618, serving as a fixed point constant across RS derivations. Phi appears in scaling laws and defines self-similar relationships in the spectra of DNA dynamics.

The DNA length constant XDNA (116) is approximately 13.6 Angstroms, representing a characteristic spatial scale derived from RS recognition principles.

The periodicity constant Pₒ(128, 158) is approximately 35.6 Angstroms, representing a fundamental DNA groove periodicity derived from RS constructs.

By incorporating Ecoh (126, 156), tauₒ, phi, XDNA (116), and Pₒ(128, 158) into the DNARP framework, the invention ensures that all simulations are grounded in RS-derived constants. These constants provide the quantitative foundation for stability, coherence, and recognition transforms, and distinguish the invention from conventional models that rely on empirical parameter fitting.

In another embodiment, the invention is applied to the simulation of circadian rhythm dynamics. Circadian rhythms are biological oscillations with an approximately twenty-four-hour period that regulate gene expression, metabolic activity, and cellular behavior. Modeling such processes requires capturing both periodicity and feedback-driven transitions in DNA dynamics.

As applied in the present invention, the DNARP state D(t)=(S(t), H(t), E(t)) is initialized to represent a baseline genetic program associated with circadian regulation. Evolution rules are defined to model oscillatory protein binding events that periodically modify sequence accessibility S(t) (192), shape conformation H(t) (194), or coherence energy E(t) (196).

The time evolution of the DNARP state is computed using the governing constructs LDNA(t), H\hat{ }DNA(t), and FDNA(E(t)). Oscillatory changes in S(t), H(t), and E(t) produce predicted time-series outputs that exhibit periodic fluctuations in gene expression intensity. The invention thus simulates circadian rhythm dynamics as recurring transitions between coherence states.

By applying parameter-free RS constructs, the invention provides a first-principles model of circadian oscillations that does not rely on empirical curve-fitting of rate constants. The results capture the phase, amplitude, and period of circadian gene regulation, demonstrating that the framework applies not only to single interaction events such as gene activation but also to longer-term oscillatory biological processes.

1. A computer-implemented method for simulating the dynamics of a biological process involving a DNA molecule, the method comprising the steps of defining, in a computer memory, an initial DNA Recognition Physics (DNARP) state D(tₒ)=(S(tₒ), H(tₒ), E(tₒ)), wherein S is a sequence component, H is a shape component, and E is an energy component, each defined according to Recognition Physics (RS) principles using RS-derived constants, defining, in the computer memory, one or more RS-based evolution rules specifying how at least one of the sequence component, shape component, or energy component changes over time in response to simulated biological interactions or stimuli, evolving, using a processor, the state D(t) from to over a time interval by iteratively solving time-dependent RS governing constructs selected from the group consisting of: a DNA Lagrangian LDNA(t), a DNA operator H\hat{ }DNA(t), and a DNA Recognition Transform FDNA(E(t)), and generating, via a computer output module, a prediction of a time course of one or more functional outputs derived from the evolved state D(t).

2. The method of claim 1, wherein the RS-derived constants include at least one of: Ecoh, tauₒ, phi, XDNA, or Pₒ, each derived parameter-free from RS axioms.

3. The method of claim 1, wherein the RS-derived constants are applied without parameter fitting, such that no adjustable empirical parameters are introduced into the simulation.

4. The method of claim 1, wherein the evolution rules comprise modification of the potential term VDNA(x;P,t) within the operator H\hat{ }DNA(t) based on a binding event or regulatory interaction.

5. The method of claim 1, wherein the energy component E(t) transitions between discrete coherence states En(t) in response to simulated excitation or relaxation processes.

6. The method of claim 5, wherein evolving the state D(t) comprises solving a time-dependent Schrödinger-like equation using H\hat{ }DNA(t) to propagate a quantum state ψ(x,t).

7. The method of claim 1, wherein structural stability is dynamically evaluated by applying LDNA(t) to the evolving sequence component S(t) and shape component H(t).

8. The method of claim 1, wherein the predicted functional output includes a gene expression rate determined by the amplitude |FDNA(E(t))|² and a base rate R(t) proportional to the energy state E(t).

9. The method of claim 1, wherein the biological process simulated is selected from the group consisting of gene regulation, response to drug exposure, response to environmental stress, circadian rhythm dynamics, and DNA repair.

10. A computational system for simulating the dynamics of a biological process involving a DNA molecule, the system comprising a processor, a memory communicatively coupled to the processor, an input module (172) configured to receive an initial DNARP state D(tₒ)=(S(tₒ), H(tₒ), E(tₒ)) and one or more RS-based evolution rules, a dynamic simulation engine (174) executed by the processor, configured to, apply the evolution rules to update components of D(t) over successive time steps, solve time-dependent RS constructs selected from the group consisting of LDNA(t), H\hat{ }DNA(t), and FDNA(E(t)) to determine the state evolution and functional output, and an output module (176) configured to present a predicted time course of functional outputs derived from the evolved state D(t).

11. The system of claim 10, wherein the dynamic simulation engine (174) is configured to solve a time-dependent Schrödinger-like equation involving the operator H\hat{ }DNA(t).

12. The system of claim 10, wherein the RS-derived constants include at least one of Ecoh, tauₒ, phi, XDNA, or Pₒ.

13. The system of claim 10, wherein the dynamic simulation engine (174) is configured to apply evolution rules without parameter fitting, such that simulation results are determined solely from RS-derived constants.

14. The system of claim 10, wherein the output module (176) is further configured to generate predictions for circadian rhythm dynamics or other oscillatory biological processes.
